SEQUENCE LISTING (2 sequences)

SEQ ID NO: 1 - DNA, 1449 bp, synthetic construct
  misc_feature 1..1449: note = Synthetic Sequence
  source 1..1449: mol_type = other DNA
taatacatgcaagtcgaacgagttctcgttgatgatcggtgcttgcaccgagattcaacatggaacgagtggcggacgggtgagtaacacgtgggtaacctgcccttaagtgggggataacatttggaaacagatgctaataccgcatagatccaagaaccgcatggttcttggctgaaagatggcgtaagctatcgcttttggatggacccgcggcgtattagctagttggtgaggtaatggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaa

SEQ ID NO: 2 - DNA, 1439 bp, synthetic construct
  misc_feature 1..1439: note = Synthetic Sequence
  source 1..1439: mol_type = other DNA
tgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagcttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaa

(19) United States
(12) Patent Application Publication
(10) Pub. No.: US 2025/0295714 A1
(43) Pub. Date: Sep. 25, 2025

(54) LACTOBACILLUS PARACASEI STRAIN OR LACTOBACILLUS PLANTARUM STRAIN DERIVED FROM HUMAN BODY, HAVING BODY FAT REDUCING ACTIVITY, AND MIXTURE COMPOSITION COMPRISING SAME

(71) Applicant: HECTO HEALTH CARE CO., LTD., Seoul (KR)

(72) Inventors: Seokjin KIM, Seoul (KR); Kum-Joo SHIN, Seoul (KR); Jongseo LEE, Gyeonggi-do (KR); Dong-Wan LEE, Seoul (KR); Eui-Cheon JEONG, Seoul (KR); Hyeji LIM, Seoul (KR); Na-Rae LEE, Seoul (KR); Tae-Jun KWON, Daegu (KR)

(21) Appl. No.: 18/859,192

(22) PCT Filed: May 25, 2023
(86) PCT No.: PCT/KR2023/007170

§ 371 Date: Oct. 23, 2024

(30) Foreign Application Priority Data
May 27, 2022 (KR) 10-2022-0065475

(51) Int. Cl. A61K 35/747; A61P 3/04; C12N 1/20; C12R 1/225

(52) U.S. Cl. CPC A61K 35/747; A61P 3/04; C12N 1/205; C12R 2001/225

(57) ABSTRACT

The present invention relates to novel lactobacillus mixture compositions having the excellent effect of preventing obesity, and use thereof. More particularly, the present invention relates to lactobacillus mixture compositions comprising Lactobacillusparacasei BCC-LP-02 (accession number KCTC14808BP) strain, and Lactobacillus plantarum BCC-LPL-53 (accession number KCTC 14809BP) strain, and use thereof.

### TECHNICAL FIELD

The present invention relates to novel strains of the genus Lactobacillus, cultures thereof, mixed strains thereof, and a body fat reduction composition comprising same, more particularly novel Lactobacillus paracasei or Lactobacillus plantarum strain, cultures thereof, mixed strains thereof, and a body fat reduction pharmaceutical composition, a dietary supplement composition, and a quasi-drug composition comprising same.

### RELATED ART

Obesity is a condition in which energy intake and expenditure are out of balance, resulting in excess energy being stored as fat, leading to abnormally high body fat and a variety of metabolic abnormalities. Obesity due to excess body fat is a major contributor to the increased risk of diabetes, cardiovascular disease, and certain cancers.

The main cause of this obesity is the accumulation of fat due to excessive calorie intake, which leads to an increase in fat cells through various mechanisms. First, fats are broken down and absorbed by an enzyme called lipase, and excess carbohydrates are broken down into sugars, which can raise blood sugar or promote differentiation into adipocytes, leading to the production of fat cells. The accumulation of fat in this process leads to obesity.

There are two main mechanisms of fat loss: inhibiting fat digestion and absorption by inhibiting the action of enzymes such as lipase, and inhibiting fat synthesis by directly inhibiting the synthesis of fat cells. Therefore, the preadipocyte 3T3L-1 has been studied in vitro to understand the cells that produce fat in vivo (Applied Biological Chemistry volume 63, Article number: 9 (2020)).

Due to the increasing number of complications arising from obesity, research on therapeutic agents that inhibit the process of obesity development is increasing both domestically and globally, but their effectiveness is limited and their side effects are significant. There are a number of medications that suppress appetite, many of which can lead to liver damage, hormonal changes, and weight regain when the medication is discontinued, which is why they are often taken for long periods of time. As the duration of administration increases and the number of cases increases, the probability of problems increases, so even if the side effects are less frequent, serious side effects cannot be overlooked, so the development of harmless and natural materials that can replace these drugs is required.

Probiotics are among the many candidates for body fat reduction being studied, and it has been reported that probiotics help prevent weight gain in mice fed a high-fat diet and significantly reduce fat loss and obesity-related biochemical markers.

Therefore, there is a need to develop safe and effective probiotic strains that are effective in inhibiting fat absorption and inhibiting cell differentiation.

With this technical background, the inventors of the present invention have isolated a novel Lactobacillus paracasei or Lactobacillus plantarum strain, confirmed its body fat reduction effect, and thus completed the present invention.

### SUMMARY OF INVENTION

It is an object of the present invention to recognize the aforementioned problems in the prior art and to provide novel Lactobacillus paracasei strains or cultures thereof.

It is an object of the present invention to provide a novel Lactobacillus plantarum strain or a culture thereof.

It is an object of the present invention to provide a body fat reduction composition comprising the strain or a culture thereof, or mixed strains or cultures thereof. It is an object of the present invention to provide a use for the preparation of a formulation for reducing body fat comprising the strain or culture thereof. It is an object of the present invention to provide a method of reducing body fat comprising the step of administering the strain or culture thereof.

It is an object of the present invention to provide a pharmaceutical composition for the prevention or treatment of obesity comprising the strain, culture thereof, or mixed strains or cultures thereof. It is an object of the present invention to provide a use for the preparation of a formulation for the prevention or treatment of obesity comprising the strain, culture thereof, or mixed strains or cultures thereof. It is an object of the present invention to provide a method of preventing or treating obesity comprising the step of administering the strain, a culture thereof, or mixed strains.

It is an object of the present invention to provide a dietary supplement composition comprising the strain, a culture thereof, or mixed strains or cultures thereof for the prevention or amelioration of obesity.

It is an object of the present invention to provide a quasi-drug comprising the strain, a culture thereof, or mixed strains or cultures thereof for the prevention or amelioration of obesity.

To accomplish the above objectives, the following solutions are provided.

One aspect of the present invention provides Lactobacillus paracasei strain BCC-LP-02 with accession number KCTC14808BP, or a culture thereof.

Another aspect of the present invention is for use in the manufacture of a composition for reducing body fat comprising Lactobacillus paracasei strain BCC-LP-02 with accession number KCTC14808BP, or a culture thereof.

Another aspect of the present invention is to provide a method of reducing body fat comprising the step of administering Lactobacillus paracasei strain BCC-LP-02 with accession number KCTC 14808BP, or a culture thereof.

One aspect of the present invention provides Lactobacillus plantarum strain BCC-LPL-53 with accession number KCTC 14809BP, or a culture thereof.

Another aspect of the present invention is for use in the manufacture of a composition for reducing body fat comprising Lactobacillus plantarum strain BCC-LPL-53 with accession number KCTC 14809BP, or a culture thereof.

Another aspect of the present invention is to provide a method of reducing body fat comprising the step of administering Lactobacillus plantarum strain BCC-LPL-53 with accession number KCTC 14809BP, or a culture thereof.

One aspect of the present invention provides a composition for reducing body fat comprising Lactobacillus paracasei strain BCC-LP-02 with accession number KCTC14808BP, or a culture thereof; and/or Lactobacillus plantarum strain BCC-LPL-53 with accession number KCTC 14809BP, or a culture thereof.

One aspect of the present invention provides a composition for reducing body fat comprising Lactobacillus paracasei strain BCC-LP-02 with accession number KCTC14808BP, or a culture thereof; and Lactobacillus plantarum strain BCC-LPL-53 with accession number KCTC 14809BP, or a culture thereof.

One aspect of the present invention provides a pharmaceutical composition for the prevention or treatment of obesity comprising Lactobacillus paracasei strain BCC-LP-02 with accession number KCTC14808BP or a culture thereof, Lactobacillus plantarum strain BCC-LPL-53 with accession number KCTC 14809BP or a culture thereof, or a mixture of the above strains or a culture thereof.

Another aspect of the present invention provides a method of preventing or treating obesity comprising administering to a patient Lactobacillus paracasei strain BCC-LP-02 with accession number KCTC 14808BP, or a culture thereof, Lactobacillus plantarum strain BCC-LPL-53 with accession number KCTC 14809BP, or a culture thereof, or a mixture of the above strains or a culture thereof.

Another aspect of the present invention provides use of Lactobacillus paracasei strain BCC-LP-02 with accession number KCTC14808BP, or a culture thereof, Lactobacillus plantarum strain BCC-LPL-53 with accession number KCTC 14809BP, or a culture thereof, or a mixture of the above strains or a culture thereof, in the preparation of an agent for the prevention or treatment of obesity.

One aspect of the present invention provides a dietary supplement composition for the prevention or amelioration of obesity comprising Lactobacillus paracasei strain BCC-LP-02 with accession number KCTC14808BP or a culture thereof, Lactobacillus plantarum strain BCC-LPL-53 with accession number KCTC 14809BP or a culture thereof, or a mixture of the above strains or a culture thereof.

One aspect of the present invention provides a quasi-drug composition for the prevention or amelioration of obesity comprising Lactobacillus paracasei strain BCC-LP-02 with accession number KCTC14808BP or a culture thereof, Lactobacillus plantarum strain BCC-LPL-53 with accession number KCTC 14809BP or a culture thereof, or a mixture of the above strains or a culture thereof.

### BRIEF DESCRIPTION OF DRAWING

FIG. 1 shows the 16S rRNA gene sequences of the novel Lactobacillus paracasei BCC-LP-02 (KCTC 14808BP) and Lactobacillus plantarum BCC-LPL-53 (KCTC 14809BP).

FIG. 2 shows the phylogenetic tree of the novel Lactobacillus paracasei BCC-LP-02(KCTC 14808BP) and Lactobacillus plantarum BCC-LPL-53 (KCTC 14809BP).

FIG. 3 shows the results of the four strains with the highest fatty acid (FA) absorption compared to the uninoculated control in an in vitro experiment, out of about 300 different species.

FIG. 4 is a picture of mucoid colonies on sucrose agar of the four selected strains confirming their ability to produce EPS.

FIG. 5 is a graph showing the lipase inhibitory activity of the four selected strains.

FIG. 6 shows a graph evaluating the ability of the four selected strains to inhibit glucose absorption using Caco-2 cells.

FIG. 7 is a graph showing the ability of the four selected strains to inhibit adipocyte differentiation in preadipocytes, 3T3-L1.

FIG. 8 is a graph showing the increase and decrease patterns of several metabolites according to incubation time of the selected strains for body fat reduction for the selection of formulation strains.

FIG. 9 is a graph showing the inhibition of preadipocyte differentiation according to the mixing ratio of the two selected formulation strains.

FIG. 10 is a graph showing the reduction in body weight gain after oral administration of probiotic complex F (LP02+LPL53) to mice for 8 weeks.

FIG. 11 is a graph showing the decrease in food efficiency after oral administration of probiotic complex F (LP02+LPL53) to mice for 8 weeks.

FIG. 12 is a graph showing the reduction in subcutaneous fat and abdominal fat (epididymal and peri-renal fat) after oral administration of probiotic complex F (LP02+LPL53) to mice for 8 weeks and post-mortem.

### DETAILED DESCRIPTION OF THE INVENTION AND PREFERRED EXAMPLES

Unless otherwise defined, all technical and scientific terms used herein shall have the same meaning as commonly understood by those skilled in the art. In general, the nomenclature used herein is well known and in common use in the art. In general, the nomenclature used herein is well known and commonly used in the art.

The present invention provides, in one aspect, the following novel strains or cultures thereof:

Lactobacillus paracasei strain BCC-LP-02 with accession number KCTC14808BP; or

Lactobacillus plantarum strain BCC-LPL-53 with accession number KCTC 14809BP.

Strains according to the present invention have been deposited with Korea Research Institute of Bioscience and Biotechnology on Dec. 6, 2021. The Lactobacillus paracasei strain with accession number KCTC14808BP is listed as BCC-LP-02 (sometimes LP-02) and the Lactobacillus plantarum strain with accession number KCTC 14809BP is listed as BCC-LPL-53 (sometimes LPL-53).

The Lactobacillus paracasei strain BCC-LP-02 with accession number KCTC14808BP contains the 16S rRNA sequence of SEQ ID NO: 1. Lactobacillus plantarum strain BCC-LPL-53 with accession number KCTC 14809BP contains the 16S rRNA sequence of SEQ ID NO: 2.

Each of the BCC-LP-02 strain and BCC-LPL-53 strain may each exhibit one or more characteristics selected from the group consisting of:

reduced fatty acid (FA) concentration;

exopolysaccharide (EPS) generation;

lipase enzyme inhibitory activity;

poor glucose absorption; and

inhibitory activity of preadipocyte differentiation.

Specifically, it can be Lactobacillus paracasei BCC-LP-02 strain or Lactobacillus plantarum BCC-LPL-53 strain with reduced fatty acid (FA) concentration, extracellular polysaccharide (EPS) production, lipase enzyme inhibitory activity, reduced glucose absorption, and inhibitory activity of preadipocyte differentiation.

With respect to cultures of the strains, “cultures” may mean cultures of each of the BCC-LP-02 and BCC-LPL-53 strains or cultures grown in culture media or broths containing the BCC-LP-02 and BCC-LPL-53 strains. The cultures may or may not comprise each of the BCC-LP-02 strain and BCC-LPL-53 strain, or both BCC-LP-02 strain and BCC-LPL-53 strain. The cultures may be liquid or solid in formulation, but are not limited thereto.

Various forms of the cultures may include, for example, concentrates, dried products, or extracts of the cultures.

The extracts can be extracted using water, an organic solvent, or the like, for example, water, lower alcohol having 1 to 4 carbon atoms, hexane, chloroform, ethyl acetate, or a mixture of solvents thereof.

In another aspect of the present invention, it relates to a body fat reduction composition comprising Lactobacillus paracasei strain BCC-LP-02 with accession number KCTC14808BP, or a culture thereof; and/or Lactobacillus plantarum strain BCC-LPL-53 with accession number KCTC 14809BP, or a culture thereof.

The present invention relates to a body fat reduction composition comprising Lactobacillus paracasei strain BCC-LP-02 with accession number KCTC 14808BP, or a culture thereof.

The present invention relates to a body fat reduction composition comprising Lactobacillus plantarum strain BCC-LPL-53 with accession number KCTC 14809BP, or a culture thereof.

The present invention relates to a body fat reduction composition comprising Lactobacillus paracasei strain BCC-LP-02 with accession number KCTC14808BP, or a culture thereof; and/or Lactobacillus plantarum strain BCC-LPL-53 with accession number KCTC 14809BP, or a culture thereof.

The mixing of BCC-LP-02 strain and BCC-LPL-53 strain may exhibit a synergistic effect in the co-culture of BCC-LP-02 strain and BCC-LPL-53 strain, which maintains a complementary relationship in the utilization of metabolome (glyceric acid, malic acid, orotic acid, lactose) and helps growth.

The BCC-LP-02 strain and the BCC-LPL-53 strain may be mixed in a ratio of, for example, 6:4 to 4:6 in terms of the number of strains, and there is no limit to the mixing ratio provided that the properties are maintained, but more specifically, the strains may be mixed in a ratio of 2:8, 3:7, 4:6, 5:5, 6:4, 7:3, or 8:2. The optimal mixing ratio of the above strains may show synergistic effects in inhibiting preadipocyte differentiation.

In another aspect, the present invention relates to a pharmaceutical composition for the prevention or treatment of obesity comprising the BCC-LP-02 strain or a culture thereof, the BCC-LPL-53 strain or a culture thereof, or a mixture of the strains or cultures thereof.

The present invention relates to a pharmaceutical composition for the prevention or treatment of obesity comprising the BCC-LP-02 strain or a culture thereof.

The present invention relates to a pharmaceutical composition for the prevention or treatment of obesity comprising the BCC-LPL-53 strain or a culture thereof.

The present invention relates to a pharmaceutical composition for the prevention or treatment of obesity comprising the BCC-LP-02 strain or a culture thereof, and the BCC-LPL-53 strain or a culture thereof.

“Prevention” means any act of inhibiting or delaying the development of the obesity or obesity-related diseases by administration of a composition according to the present invention. “Treatment or amelioration” means any action that improves or favorably changes the condition of obesity or obesity-related conditions.

The obesity-related diseases may include, but are not limited to, diabetes, hyperlipidemia, heart disease, stroke, atherosclerosis, fatty liver, and other metabolic diseases.

When administered to an individual, the composition may reduce the rate of weight gain, reduce food efficiency, or reduce subcutaneous fat and abdominal fat (epididymal fat, peri-renal fat).

The composition may have the effect of inhibiting absorption of fatty components, inhibiting absorption of carbohydrates, and/or inhibiting fat differentiation in small intestinal cells or the digestive tract. The pharmaceutical composition may be prepared in unit dose form or in multi-dose containers by formulation with pharmaceutically acceptable carriers and/or excipients, in accordance with methods readily practiced by a person of ordinary skill in the art. The formulation may be in the form of a solution, suspension, or emulsion in an oil or aqueous medium, or in the form of an excipient, powder, granule, tablet, capsule, or gel (e.g., hydrogel), and may further comprise a dispersant or stabilizing agent.

Pharmaceutically acceptable carriers may include, but not limited to, lactose, glucose, sucrose, sorbitol, mannitol, starch, acacia, gum, calcium phosphate, alginate, gelatin, calcium silicate, microcrystalline cellulosepolyvinyl pyrrolidone, cellulose, water, syrup, methyl cellulose, methylhydroxybenzoate, propyl hydroxybenzoate, talc, magnesium stearate, and mineral oil, which are customarily utilized in the course of preparation. In addition to the above ingredients, lubricants, wetting agents, sweeteners, flavoring agents, emulsifiers, suspending agents, or preservatives may be further included.

The pharmaceutical composition may be administered orally or parenterally and may be used in the form of a conventional pharmaceutical preparation. In other words, the pharmaceutical composition of the present invention can be administered in a variety of oral and parenteral dosage forms in actual clinical administration, wherein it is formulated using commonly used diluents or excipients such as fillers, extenders, binders, wetting agents, disintegrants, surfactants, and the like. Solid preparations for oral administration include tablets, pills, acid preparations, granules, capsules, and the like, which are prepared by mixing a herbal extract or herbal ferment with at least one excipient, such as starch, calcium carbonate, sucrose or lactose, gelatin, and the like. In addition to simple excipients, lubricants such as magnesium stearate talc are also used. Liquid preparations for oral administration include suspensions, oral solutions, emulsions, and syrups, which may include a variety of excipients, such as wetting agents, sweeteners, flavors, and preservatives, in addition to the commonly used simple diluents such as water and liquid paraffin. Formulations for parenteral administration include sterile aqueous solutions, non-aqueous solutions, suspensions, emulsions, lyophilizations, and suppositories. Non-aqueous, suspending solvents can include propylene glycol, polyethylene glycol, vegetable oils such as olive oil, and injectable esters such as ethylolates. The base of the suppository can include Witepsol, Macrogol, Twin 61, cacao paper, laurin paper, glycerol, gelatin, etc.

The concentration of the active ingredient included in the composition can be determined by considering the purpose of treatment, the condition of the patient, the duration of need, etc. and is not limited to a specific range. The pharmaceutical composition of the present invention is administered in a pharmaceutically effective amount. As used in the present invention, a “pharmaceutically effective amount” refers to an amount sufficient to treat a condition with a reasonable benefit/risk ratio applicable to medical treatment, and the effective dosage level may be determined based on factors including the type and severity of the patient's condition, the activity of the drug, the sensitivity to the drug, the timing and route of administration, and the rate of drug elimination, the duration of treatment, concomitant medications, and other factors well known in the medical field. The pharmaceutical composition of the present invention may be administered as an individual therapeutic agent, or in combination with agents for treating diseases caused by other pollutants or for improving skin aging, and may be administered simultaneously, separately, or sequentially with conventional agents, and may be administered singly or in multiple doses. Taking all of these factors into account, it is important to administer an amount that will produce the maximum effect in the least amount without side effects, which can be readily determined by those skilled in the art. The effective amount may vary depending on the patient's age, gender, condition, weight, absorption of the active ingredient in the body, inactivation rate, excretion rate, type of disease, concomitant medications, route of administration, severity of obesity, gender, weight, age, etc.

In another aspect, the present invention relates to a dietary supplement composition comprising the BCC-LP-02 strain or a culture thereof, the BCC-LPL-53 strain or a culture thereof, or a mixture of the strains or cultures thereof, for the prevention or amelioration of obesity.

The present invention relates to a dietary supplement composition comprising the BCC-LP-02 strain or cultures thereof for the prevention or amelioration of obesity.

The present invention relates to a dietary supplement composition comprising the BCC-LPL-53 strain or cultures thereof for the prevention or amelioration of obesity.

The present invention relates to a dietary supplement composition for the prevention or amelioration of obesity comprising the BCC-LP-02 strain or a culture thereof, and the BCC-LPL-53 strain or a culture thereof.

A dietary supplement is a food that has high medical effects and has been processed to efficiently perform bioregulatory functions in addition to providing nutrition. Dietary supplements can be manufactured in various forms, such as tablets, capsules, powders, granules, liquids, and pills, to achieve useful effects in the prevention or amelioration of obesity.

The dietary supplement composition may be formulated as a food product, in particular a functional food product. The functional food of the present invention includes ingredients that are conventionally added during food manufacturing and may include, for example, proteins, carbohydrates, fats, nutrients, and seasonings. For example, if formulated as a beverage, it may contain natural carbohydrates or flavoring agents as additional ingredients in addition to the active ingredients. Preferably, the natural carbohydrates are monosaccharides (e.g., glucose, fructose, etc.), disaccharides (e.g., maltose, sucrose, etc.), oligosaccharides, polysaccharides (e.g., dextrin, cyclodextrin, etc.), or sugar alcohols (e.g., xylitol, sorbitol, erythritol, etc.). The flavoring agents can be natural (e.g., thaumatin, stevia extract, etc.) and synthetic (e.g., saccharin, aspartame, etc.).

There are no specific restrictions on the types of dietary supplements. These include dairy products, beverages, tea drinks, alcoholic beverages, and vitamin complexes, and can include all health foods in the usual sense.

It may further include various nutrients, vitamins, electrolytes, flavoring agents, coloring agents, pectic acid and its salts, alginic acid and its salts, organic acids, protective colloidal thickeners, pH regulators, stabilizers, preservatives, glycerin, alcohol, carbonation agents used in carbonated beverages, and the like.

In another aspect, the present invention relates to a quasi-drug composition for the prevention or amelioration of obesity comprising the BCC-LP-02 strain or a culture thereof, the BCC-LPL-53 strain or a culture thereof, or a mixture of the strains or cultures thereof.

The present invention relates to a quasi-drug composition comprising the BCC-LP-02 strain or cultures thereof for the prevention or amelioration of obesity.

The present invention relates to a quasi-drug composition comprising the BCC-LPL-53 strain or cultures thereof for the prevention or amelioration of obesity.

The present invention relates to a quasi-drug composition for the prevention or amelioration of obesity comprising the BCC-LP-02 strain or a culture thereof, and the BCC-LPL-53 strain or a culture thereof.

A quasi-drug is a preparation used for the prevention or amelioration of obesity, other than a preparation used for the purpose of pharmacologically affecting the structure or function of a human or animal.

When used as a quasi-drug, the BCC-LP-02 strain or a culture thereof, the BCC-LPL-53 strain or a culture thereof, or mixed strains or cultures thereof, may be used in combination with other quasi-drug components, and may be used appropriately according to conventional methods. The amount of the active ingredients may be suitably determined depending on the intended use (preventive, health or therapeutic treatment).

### EXAMPLES

The present invention will now be described in more detail with reference to the following examples. These examples are intended solely to illustrate the present invention, and it will be apparent to one of ordinary skill in the art that the scope of the present invention is not to be construed as limited by these examples.

### Example 1. Isolation of Lactobacillus Paracasei BCC-LP-02 and Lactobacillus Plantarum BCC-LPL-53 Strains

To select the novel strains for Koreans, feces and breast milk from healthy Korean babies and adults were used as samples. One gram of sample was diluted 10-fold serially in sterile saline, and 0.1 mL of the dilution was smeared onto MRS solid medium and incubated for 3 days under anaerobic conditions. The resulting single colonies were purely cultured in MRS liquid medium at 37° C. for 18 hours.

### Example 2. Sugar Availability in Lactobacillus Paracasei BCC-LP-02 and Lactobacillus Plantarum BCC-LPL-53 Strains

To analyze the sugar availability of the selected strains, the API 50 CH kit (BioMerieux, Lyon, France) was used to determine the availability of 49 carbon sources. Suspensions were prepared by adjusting the colonies of strains cultured on MRS solid media to a turbidity of 2 MacFarland. Turbidified API 50 CHL medium was dispensed in 120 μL into API 50CH strip tubes and 2 drops of mineral oil was added to the couples and incubated at 37° C. for 24 and 48 hours to determine the sugar utilization pattern. Identification results were verified by using the API web program (https://apiweb.biomerieux.com).

Identification results showed that it was 99.9% identical to Lactobacillus paracasei and Lactobacillus plantarum, as shown in Table 1.

TABLE 1
Lactobacillus paracasei ssp paracasei 1-99.9%
Lactobacillus platarum1 -99.9%

| No. | Carbohydrate | LP-02 |
|---|---|---|
| 1 | Control | |
| 2 | Glycerol | |
| 3 | Eryth [symbol] tol | |
| 4 | D-Arabinose | |
| 5 | L-Arabinose | + |
| 6 | D-Ribose | |
| 7 | D-Xylose | |
| 8 | L-Xylose | |
| 9 | D-Ado [symbol] lo [symbol] | |
| 10 | [symbol] Methyl- D-xylopyr [symbol] side | + |
| 11 | D- [symbol] | + |
| 12 | D-Glucose | + |
| 13 | D-F [symbol] tose | + |
| 14 | D-M [symbol] ose | |
| 15 | L-S [symbol] bose | |
| 16 | L-R [symbol] ose | |
| 17 | [symbol] | |
| 18 | [symbol] | + |
| 19 | D-M [symbol] nn [symbol] | |
| 20 | D-Sorbitol | |
| 21 | α Meth [symbol] yl- D-mannopyra [symbol] side | + |
| 22 | α Melt [symbol] yl- D-g [symbol] copyra [symbol] side | + |
| 23 | N-Acetylglucosamine | + |
| 24 | Am [symbol] ygd [symbol] | + |
| 25 | Arbutin | + |
| 26 | Esc [symbol] lin | + |
| 27 | Sa [symbol] cin | + |
| 28 | D-Co [symbol] b [symbol] | + |
| 29 | D-Maltose | + |
| 30 | D-Lac [symbol] ose | |
| 31 | D- [symbol] | + |
| 32 | D- [symbol] | + |
| 33 | D-Tr [symbol] | + |
| 34 | Inulin | + |
| 35 | D-Me [symbol] | |
| 36 | D-Ra [symbol] | |
| 37 | Starch | |
| 38 | Glycogen | |
| 39 | Xy [symbol] ol | + |
| 40 | G [symbol] | + |
| 41 | D- [symbol] ose | |
| 42 | D-Ly [symbol] | + |
| 43 | D- [symbol] s [symbol] | |
| 44 | D- [symbol] ose | |
| 45 | L-F [symbol] | |
| 46 | D-Ar [symbol] ol | |
| 47 | L-Ar [symbol] ol | + |
| 48 | Potassium gluc [symbol] | |
| 49 | Potassium 2-ketogluconate | |
| 50 | Potassium [symbol] -ketogluconate | |
| No. | Carbohydrate | LPL-53 |
| 1 | Control | |
| 2 | Glycerol | |
| 3 | Eryth [symbol] tol | |
| 4 | D-Arabinose | + |
| 5 | L-Arabinose | + |
| 6 | D-Ribose | |
| 7 | D-Xylose | |
| 8 | L-Xylose | |
| 9 | D-Ado [symbol] lo [symbol] | |
| 10 | [symbol] Methyl- D-xylopyr [symbol] side | + |
| 11 | D- [symbol] | + |
| 12 | D-Glucose | + |
| 13 | D-F [symbol] tose | + |
| 14 | D-M [symbol] ose | |
| 15 | L-S [symbol] bose | |
| 16 | L-R [symbol] ose | |
| 17 | [symbol] | |
| 18 | [symbol] | + |
| 19 | D-M [symbol] nn [symbol] | + |
| 20 | D-Sorbitol | + |
| 21 | α Meth [symbol] yl- D-mannopyra [symbol] side | |
| 22 | α Melt [symbol] yl- D-g [symbol] copyra [symbol] side | + |
| 23 | N-Acetylglucosamine | + |
| 24 | Am [symbol] ygd [symbol] | + |
| 25 | Arbutin | + |
| 26 | Esc [symbol] lin | + |
| 27 | Sa [symbol] cin | + |
| 28 | D-Co [symbol] b [symbol] | + |
| 29 | D-Maltose | + |
| 30 | D-Lac [symbol] ose | + |
| 31 | D- [symbol] | + |
| 32 | D- [symbol] | + |
| 33 | D-Tr [symbol] | |
| 34 | Inulin | + |
| 35 | D-Me [symbol] | + |
| 36 | D-Ra [symbol] | |
| 37 | Starch | |
| 38 | Glycogen | |
| 39 | Xy [symbol] ol | + |
| 40 | G [symbol] | + |
| 41 | D- [symbol] ose | |
| 42 | D-Ly [symbol] | |
| 43 | D- [symbol] s [symbol] | |
| 44 | D- [symbol] ose | |
| 45 | L-F [symbol] | |
| 46 | D-Ar [symbol] ol | |
| 47 | L-Ar [symbol] ol | + |
| 48 | Potassium gluc [symbol] | |
| 49 | Potassium 2-ketogluconate | |
| 50 | Potassium [symbol] -ketogluconate | |

[symbol]

indicates data missing or illegible when filed

### Example 3. Identification of Strains: 16S rRNA Sequencing

To identify the strains, 16S rRNA sequencing was requested to Solgent (Daejeon, Korea). DNA was extracted from 1 mL of the pure culture of the strain using the Wizard genomic DNA purification kit (Promega, USA), and the extracted DNA was subjected to PCR with primers 27F (AGAGTTTGATCMTGGCTCAG) and 1492R (TACGGYTACCTTGTTACGACTT) of the 16S rRNA sequence region as template for DNA sequencing (Solgent). Based on the sequencing results, the homology was compared with other standard strains registered in NCBI's Genebank database using BLAST.

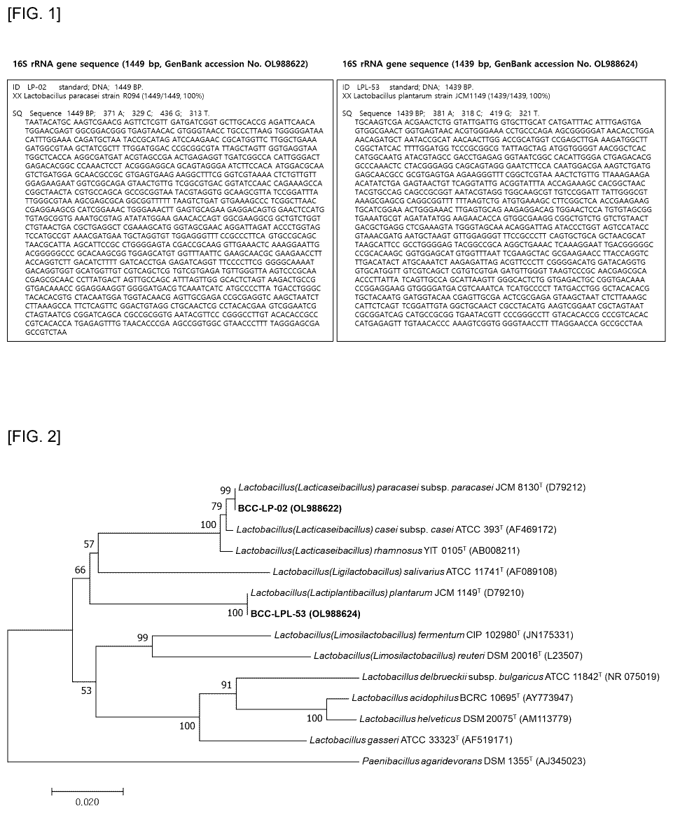

Based on sequence analysis, the 16S rRNA sequences of SEQ ID Nos: 1 and 2 (FIG. 1) were identified, and the inventors of the present invention have named LP-02 and LPL-53 lactic acid bacteria as Lactobacillus paracasei BCC-LP-02 and Lactobacillus plantarum BCC-LPL-53, respectively, and each of the lactic acid bacteria exhibited 100% homology to Lactobacillus paracsei R094T and to Lactobacillus plantarum subsp. plantarum JCM 1149T. Homology was analyzed and a phylogenetic tree was obtained using the Mega 7 program (FIG. 2)). Hereby, the inventors have identified the strains as novel strains of Lactobacillus paracasei and Lactobacillus plantarum, named Lactobacillus paracasei BCC-LP-02 and Lactobacillus plantarum BCC-LPL-53, and deposited them in Korea Research Institute of Bioscience and Biotechnology on Dec. 6, 2021 (Accession numbers KCTC 14808BP and 14809BP). In addition, the sequences of the strains have been deposited in the GenBank of the National Center for Biotechnology Information (NCBI) under accession Nos. OL988622 (BCC-LP-02) and OL988624 (BCC-LPL-53).

### Example 4. Preparation of Inactivated Selected Probiotics and Freeze-dried Powder

To perform the in vitro experiments of the selected probiotics, the lactic acid bacteria were cultured in MRS medium for 18 hours and then centrifuged at 3600 rpm for at least 15 min to obtain the bacteria, removing the supernatant and washing once with an equivalent volume of 1×PBS. The washed bacteria were concentrated by adding 1×PBS equal to 1/10 of the volume of medium in the culture and indirectly sterilized using an autoclave at 121° C. for 15 minutes.

Sterilized inactivated probiotics were frozen for 24 hours in an ultra-low freezer at −80° C. and then lyophilized for 48 hours in a freeze-dryer. The lyophilized powder was collected, refrigerated, and used in the experiments.

### Example 5. Evaluation of Fatty Acid (FA) Absorption by Strains In Vitro and Determination of Their Ability to Produce Exopolysaccharides

It can be easily measured by absorbance (OD 570 nm) using the principle that fatty acids present in the sample are converted to CoA derivatives, which are oxidized to produce intermediates (H₂O₂) that induce color development in the probe.

Strains that reduce fatty acids in the medium were selected for their ability to absorb fatty acids based on the principle that when absorbed into the body, they can reduce the concentration of fatty acids (FA) dissolved in the gut fluid content, thereby reducing the amount of fatty acids absorbed into the body and thus inhibiting fat production.

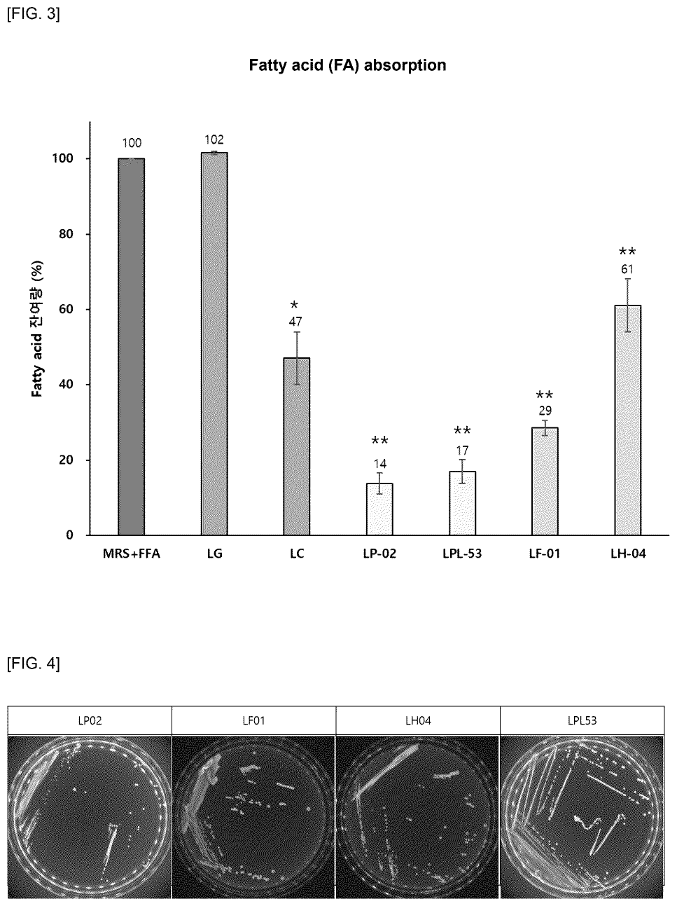

Strains isolated and preserved from the gut environment were inoculated on MRS agar or MRS (+0.05% cysteine) agar and incubated anaerobically at 37° C. in an anaerobic chamber (WHITLEY A35 Workstation, Labconsult). After 48 hours of incubation, single colonies were inoculated (using E-tubes) into 1 mL MRS or MRS (+0.05% cysteine) broth and incubated anaerobically at 37° C. for 18 to 20 hours. E-tubes containing 900 μL of MRS broth containing 0.5% (w/v) Brij58 and 0.25 mM sodium palmitate were inoculated with 100 μL of the above cultures and incubated anaerobically at 37° C. for 24 hours. The cultures were centrifuged (13,000 rpm, 1 min, 4° C.) and the supernatant was collected to determine the residual fatty acid (FA) concentration remaining in 10 μL of the supernatant. The amount of fatty acids in the supernatant was calculated by measuring the absorbance at 570 nm using the EnzyChrom™ Free Fatty Acid Assay Kit (Bio-Assay Systems, USA). The amount of fatty acids in the strain cultures was calculated with the quantitation curve of the standard substance and the ability of the strain to reduce the fatty acid concentration compared to the uninoculated control (negative control, NC) was calculated and shown in FIG. 3.

From the measurements, four strains were selected from different species that best reduced fatty acids (FAs) in the medium out of over 300 different strains of high-type strains. The results are shown with graphs of FIG. 3.

As shown in FIG. 3, the four selected strains had fatty acid absorption ranging from a low of 39% to a high of 86%, with Lactobacillus paracasei BCC-LP-02 having the highest absorption at 86%.

Exopolysaccharide (EPS) is a polysaccharide secreted and accumulated by microorganisms during metabolism, and is a primary or secondary metabolite that forms a plasma membrane around the cell wall or exists as a mucilage outside the cell wall. EPS produced by lactic acid bacteria has proven its efficacy as a natural stabilizer in fermented dairy products and has recently been studied as a functional material for various physiological functions. Immune-related functions have been reported, and other benefits include body fat reduction and cholesterol lowering. When lactic acid bacteria consume sugars and produce EPS, they are converted into polysaccharides that are poorly absorbed to the body and excreted, which can compete with conventional sugars and have a positive effect on calorie absorption. In the present invention, the ability of the strains with superior fatty acids to produce EPS was validated to identify additional fat loss-related functions. To confirm the ability to produce EPS, the selected strains were streaked on sucrose agar (1% tryptone, 0.5% yeast extract, 0.5% dipotassium phosphate, 0.5% diammonium citrate, 5% sucrose, 15% agar, pH 7.0) and incubated for 48 hours at 37° C., anaerobic conditions, and the colonies were checked for viscosity, and all four selected strains showed viscous mucoid colonies (FIG. 4).

### Example 6. Lipase Enzyme Inhibitory Activity

Pancreatic lipase is a lipolytic enzyme that breaks down triglycerides into 2-monoacylglycerol (2-) and fatty acids, and is responsible for breaking down 50% to 70% of ingested fat. Increased lipase activity allows the body to absorb more of the consumed fat in the body, increasing the accumulation of fat cells in the body. Inhibiting the activity of pancreatic lipase reduces the breakdown of fats absorbed from food, which in turn reduces the absorption of fat in the body, which in turn reduces calorie intake, resulting in weight loss.

Lipase inhibitory capacity was assessed by measuring the concentration of fatty acids (FA) finally degraded after a certain amount of time of reaction of the selected strains with lipid (triglyceride) in a lipase-treated mixture to evaluate the extent to which the lipase activity is inhibited during treating strains.

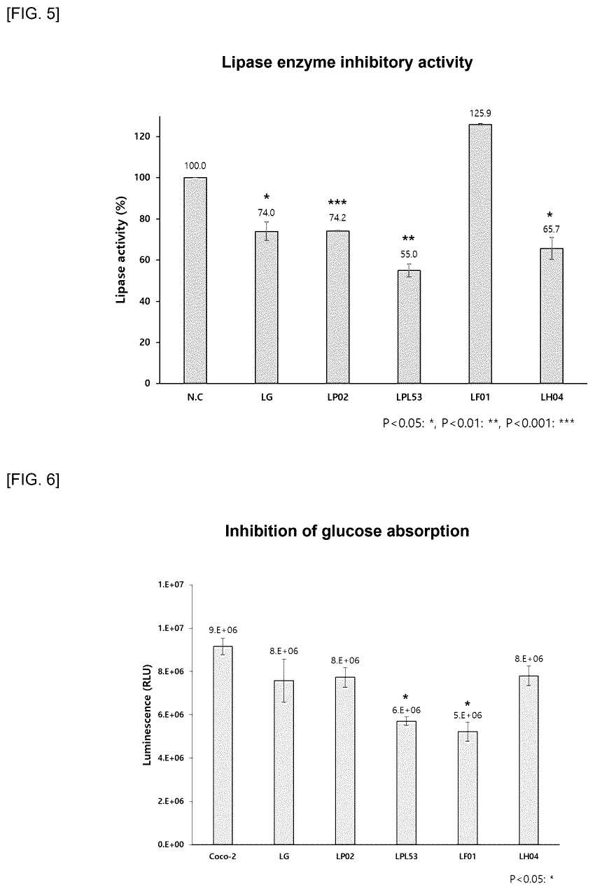

The specific test methods are as follows. A lipid solution was prepared by adding Triolein (80 mg), Lecithin (10 mg), and Taurocholic acid (5 mg) to 9 mL of TES buffer (0.1 M TES, 0.1 M NaCl, pH 7.0, Biosaesang, South Korea) and a solution of Pancreatic Lipase (500 units/mg) diluted to 10 units/mL was prepared. 100 ul of solution with heat-killed bacteria was mixed with 100 ul of lipid solution and 50 ul of lipase solution (10 units) and the mixture is reacted in a heat-block at 37° C. for 30 min. Degraded fatty acids (FA) after the reaction was measured using the EnzyChrom™ Free Fatty Acid Assay Kit (Bio-Assay Systems, USA). The lipase inhibitory activity of the four selected strains was compared to the amount of free fatty acids (FA) in the mixture without strain treatment (control) (FIG. 5).

LPL-53 strain showed the best inhibition of lipase activity at approximately 45%, followed by LH-04 and LP02 strains with 34.3% and 25.8% inhibition, respectively. LF-01 strain did not show inhibition of lipase activity. The three strains that showed lipase activity were comparable to or higher than the inhibitory capacity of the LG strain, a lactic acid bacterium known for its body fat reduction.

### Example 7. Evaluation of Inhibition of Glucose Absorption

During the digestion of food, carbohydrates are usually finally broken down into glucose, a simple sugar, which is absorbed in the intestines. The glucose that is absorbed is used as a source of energy, but the excess glucose is not absorbed and combines with fatty acids to create fat cells, leading to obesity. In the present invention, Caco-2 (human epithelial) cells, human-derived intestinal cells, purchased from the American Type Culture Collection (ATCC), were used to evaluate glucose-lowering capacity by treating the intestinal cells with the selected strains and measuring the sugar absorption of the cells. The specific methods are as follows. Caco-2 cells were cultured in MEM (Gibco, USA) medium containing 10% fetal bovine serum (FBS, Gibco, USA). Cells were cultured in 96 well plates and cultured for at least 2 days until 100% confluence, and then cultured for 15 days with medium changes every 2 days until they formed a 3D layer of cells. After completion of the culture, serum and glucose-free DMEM (ATCC, USA) medium (ATCC, USA) supplemented with the selected strain diluted to 1×10⁷ CFU/mL was treated with Caco-2 cells and incubated for 6 hours at 37° C. in a 5% CO₂ environment. After 6 hours, the strains and medium were removed, washed with 1×PBS, and the glucose absorption of the cells was measured with the Glucose uptake-Glo assay kit (Promega, USA).

The results are shown in FIG. 6. Among the four selection strains, it was found that the sugar absorption of cells treated with strains BCC-LPL-53 and BCC-LF-01 was significantly reduced compared to that of control cells treated with nothing. BCC-LF-01 showed 43% inhibition compared to control and BCC-LPL-53 showed 38% inhibition compared to control.

### Example 8. Inhibitory Activity of Preadipocyte Differentiation

1) Induction of 3T3-L1 adipocyte differentiation

3T3-L1 cells, mouse embryonic fibroblasts, purchased from the American Type Culture Collection (ATCC), were cultured in DMEM (ATCC, USA) medium containing 10% fetal bovine serum (FBS, Gibco, USA) in a 5% CO2 incubator at 37° C.

The process of differentiating 3T3-L1 mouse embryonic fibroblasts into adipocytes is as follows. Cells at 120% confluence 2 days (day 2) after the 3T3-L1 cells exhibit 100% confluence (day 0) were used. After removing the existing medium of cultured 3T3-L1 cells, they were cultured in DMEM (differentiation medium, MDI) containing 10% FBS, 1% P/S, 1 μg/mL insulin (Sigma, USA), 0.5 mM 3-isobutyl-1-methlxanthine (IBMX, Sigma, USA), and 1 μM dexamethasone (Sigma, USA). Two days after the differentiation medium treatment (day 4), the cells were exchanged for DMEM containing 10% FBS and 1 μg/mL insulin (insulin medium). Two days later (day 6), they were exchanged for existing DMEM medium containing only 10% FBS (stabilization medium). Four days after the stabilization medium change (day 10), at least 90% of the 3T3-L1 cells were considered fully differentiated into adipocytes when they formed lipid droplets.

2) Evaluation of inhibition of fat accumulation in 3T3-L1 adipocytes

At the time of addition of differentiation medium to cultured 3T3-L1 adipocytes (day 0), heat-treated/lyophilized powder of each test strain was added together at 1 mg/ml (0.1%). After 2 days of differentiation medium treatment, the strain and differentiation medium were removed, and the strain was differentiated by the differentiation induction method described above with insulin medium and stabilization medium changes for a total of 10 days.

After completion of differentiation, the cultures of 3T3-L1 preadipocyte (maturing 3T3-L1preadipocyte) with lipid droplets were washed twice with 1×PBS to remove the culture medium, and 10% formalin (Biosesang, South Korea) was added for 30 min to fix the cells. After formalin removal, cells were fixed for an additional 5 minutes by treating with 60% isopropanol. After removing isopropanol, fixed cells were treated with a staining reagent diluted with 0.5% Oil-Red O (Sigma, USA) and D.W. in a 3:2 ratio and stained for 20 minutes. After removing the staining reagent, the stained lipid droplets were washed twice with 1×PBS and observed under the microscope.

Measurement of differentiation was performed by treating the stained cells with Isopropanol 100% (Duksan, South Korea) to free the staining reagent, and then measuring the absorbance at 490 nm using a microplate reader.

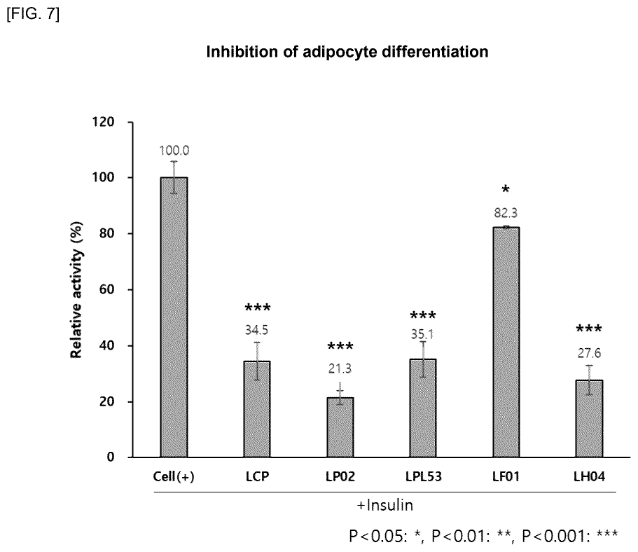

The result (FIG. 7) is shown in the illustration of the degree of adipocyte differentiation. As shown in FIG. 7, compared to the control group, the group treated with inactivated BCC-LP02 showed the highest inhibition of adipocyte differentiation at 78.7%, followed by BCC-LH-04, BCC-LPL-53, and BCC-LF-01 to the extent of 72.4%, 63.9%, and 17.7%, respectively. The first three selected strains, except for BCC-LF-01, showed similar or better inhibition than Lactobacillus complex (LCP), a commercially available functional strain for fat loss.

### Example 9. Confirmation of Metabolically Characteristics of the Selected Strains for Body Fat Reduction and Determination of Candidates

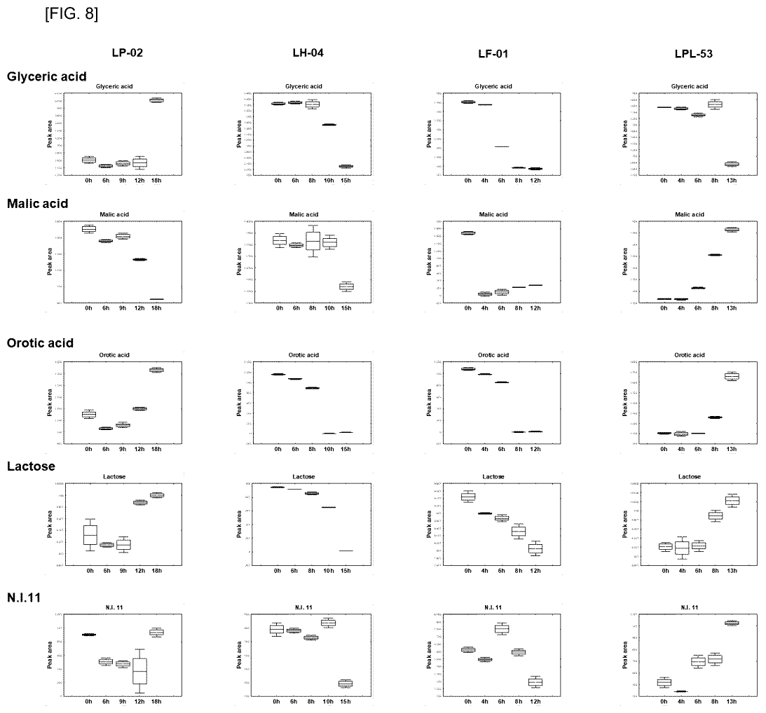

While synergy between strains were demonstrated by identifying metabolic traits used by lactic acid bacteria in culture, the metabolic traints of the selected strains were analyzed depending on a function of incubation time in order to set up formulations. The brief test methods are as follows. The four selected strain candidates for body fat reduction were cultured under anaerobic conditions at 37° C. for at least 18 hours, and five time points (Oh, mid-exponential phase, late exponential phase, early stationary phase and stationary phase) were selected for each strain's growth curve, and the metabolome of the centrifuged supernatant was analyzed using LC-MS/MS. The results are shown in FIG. 8.

As a result, a total of 74 metabolomes, including organic acids, amino acids, and carbohydrates, were identified, with significant differences between strains in five metabolomes (glyceric acid, malic acid, orotic acid, lactose, and N.I. 11 metabolite), as shown in FIG. 8 above. Analyzing the interrelationships of metabolome utilization among the four selected strains, as shown in Table 2, BCC-LPL-02 and BCC-LPL-53 showed mutual and neutral relationships with each other in all four metabolomes, while all other strains showed competition in at least one metabolome. In addition, BCC-LF-01 and BCC-LH-04 showed competition in all four metabolomes.

Therefore, among the four selected strains, BCC-LPL-02 and BCC-LPL-53 were selected as the formulation strains because of their complementary relationship through metabolomic analysis in co-culture.

TABLE 2

| Glyceric acid | LP02 | LH04 | LF01 | LPL53 |
|---|---|---|---|---|
| LP02 | | Feeding | Feeding | Feeding |
| LH04 | | | Competition | Competition |
| LF01 | | | | Competition |
| LPL53 | | | | |

| Lactose | LP02 | LH04 | LF01 | LPL53 |
|---|---|---|---|---|
| LP02 | | Feeding | Feeding | Neutral |
| LH04 | | | Competition | Feeding |
| LF01 | | | | Feeding |
| LPL53 | | | | |

| Malic acid | LP02 | LH04 | LF01 | LPL53 |
|---|---|---|---|---|
| LP02 | | Competition | Competition | Feeding |
| LH04 | | | Competition | Feeding |
| LF01 | | | | Feeding |
| LPL53 | | | | |

| Orotic acid | LP02 | LH04 | LF01 | LPL53 |
|---|---|---|---|---|
| LP02 | | Feeding | Feeding | Neutral |
| LH04 | | | Competition | Feeding |
| LF01 | | | | Feeding |
| LPL53 | | | | |

### Example 10. Setting up a Selected Animal Test Candidate Group Formulation

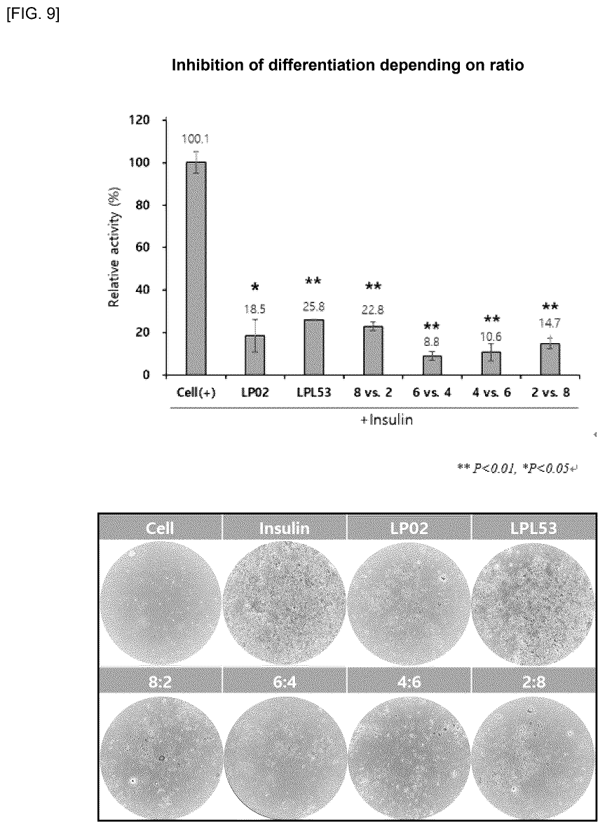

To determine the animal test formulation ratio for BCC-LP-02 and BCC-LPL-53, which have a complementary relationship in metabolomic analysis, the heat-killed freeze-dried powders of these two strains were formulated individually and in various ratios according to Example 4. These formulations were then applied to 3T3-L1 preadipocytes induced to differentiate with insulin, and the degree of inhibition was assessed. BCC-LP-02 and BCC-LPL-53 were treated in ratios of 8:2, 6:4, 4:6, and 2:8, respectively, and the results are shown in FIG. 9.

The results showed better differentiation inhibition in the group treated with formulation than in the group treated with individual strain, and among the formulations, cells treated with BCC-LP-02 and BCC-LPL-53 at a ratio of 6:4 and 4:6 showed a significantly lower differentiation rate of 8.8% and 10.6%, respectively, compared to differentiated cells.

Table 3 summarizes the selection process by combining the results from Example 1 to 9. Overall, BCC-LPL-53 showed the best body fat reduction efficacy, and BCC-LP-02 was selected based on the metabolomic analysis results, and the final animal test candidate was selected through a ratio test.

TABLE 3

| Biomarker for body fat reduction / | | | | | | |
|---|---|---|---|---|---|---|
| strain and score | | | | | | |
| (1~4, 0: no effect) | | LP-02 | LP-L53 | LF-01 | LH-04 | Remarks |
| in | 1. FA absorption | 2 | 4 | 3 | 1 | |
| vitro | 2. Lipase inhibition | 2 | 4 | 0 | 3 | |
| | 3. EPS generation | 1 | 1 | 1 | 1 | |
| | 4. Glucose absorption | 0 | 4 | 3 | 0 | |
| | inhibition | | | | | |
| | 5. Preadipocyte (3T3L1) | 4 | 2 | 1 | 3 | |
| | differentiation inhibition | | | | | |
| | 6. Total score | 9 | 15 | 10 | 8 | |
| | 7. Feeding | 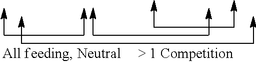 | | | | |
| | 8. Preadipocyte (3T3L1) | 8 | 2 | | | |
| | formulation test | 6 | 4 | | | |
| | | 4 | 6 | | | |
| | | 2 | 8 | | | |

### Example 11. Body Fat Reduction Experiment in Test Animals

To test the effectiveness of body fat reduction in mice, 5-week-old C57BL/6J (Charles River Japan) mice were acclimated for 1 week and then divided into 3 groups. Group 1 received a normal diet (ND), group 2 received a high-fat diet (HFD, 60 Kcal % fat diet, D12492), and group 3 received a high-fat diet plus probiotic complex F (LP02+LPL53, 109 CFU/mouse) orally 5 times per week. Body weight and food intake were checked twice weekly, and after 8 weeks of treatment, the animals were necropsied to measure the weight and size of adipose tissue.

### 1) Reduction in Mouse Weight Gain Rate

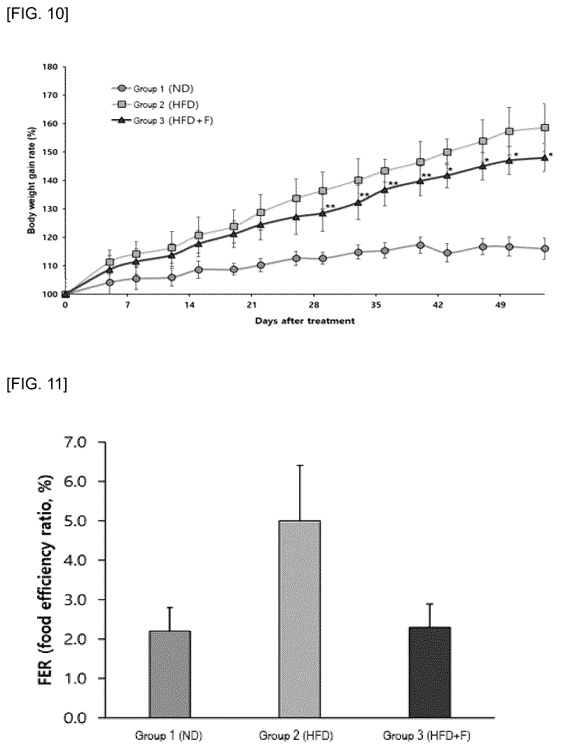

Using week 0 weight as 100%, different weight gain rates were measured at different times and plotted in FIG. 10. As shown in FIG. 10, Group 3 (HFD+F) was compared to Group 2 (HFD), with weight gain over time measured from week 1 to using week 0 weight as 100%. As shown in FIG. 10, Group 3 (HFD+F) showed a difference in weight gain from 1 week onward compared to Group 2 (HFD). At week 6, the weight gain of Group 3 was 139.86%, while the weight gain of Group 2 was 146.56%, indicating that Group 3 gained 6.7% less weight than Group 2. At week 8, Group 3 had a weight gain of 148.06%, an 8.2% weight gain compared to week 6, while Group 2 had a weight gain of 158.65%, a 12.1% weight gain compared to week 6. Specifically, at week 8, Group 3 had 10.6% less weight gain than Group 2. As shown in Table 4 below, it was found that the 8-week weight gain of Group 3 was 11.12, while the 8-week weight gain of Group 2 was 13.40, representing a difference of at least 17%.

Thus, when comparing Group 2 with a high-fat diet to Group 3 with a high-fat diet plus probiotic complex F (LP02+LPL53), it can be seen that the difference in weight gain begins to show up after one week and becomes significantly greater over time.

TABLE 4

| | Group 1 | Group 2 | Group 3 |
|---|---|---|---|
| | (ND) | (HFD) | (HFD + F) |
| Weight gain rate at week 6 (%) | 117.334 | 146.56 | 139.86 |
| Weight gain rate at week 6 (%) | 116.09 | 158.65 | 148.06 |
| 8-week weight gain (%) | 3.68 | 13.40 | 11.12 |

### 2) Decrease in Food Efficiency

Weight gain, food intake, and food efficiency over the 8 weeks are shown in Table 5.

TABLE 5

| | Group 1 | Group 2 | Group 3 |
|---|---|---|---|
| | (ND) | (HFD) | (HFD + F) |
| Weight gain (g) | 3.68 | 13.40 | 11.12 |
| Food intake (g/mouse) | 167.04 | 273.12 | 504.18 |
| Food efficiency (weight | 2.20 | 4.90 | 2.21 |
| gain/food intake) | | | |

As shown in Table 5 above, Group 3 actually lost 2.3 grams of body weight despite a 1.8-fold increase in food intake per mouse compared to Group 2. Therefore, when the food efficiency was calculated from the weight gain and food intake, it was 4.90 in Group 2, but 2.21 in Group 3, which is about 55% less than in Group 2. This result was similar to the food efficiency of 2.20 in Group 1. As can also be seen in FIG. 11, the probiotic complex F (LP02+LPL53) treatment group did not gain weight despite the increased food intake, suggesting that it contributes to weight loss.

3) Reduction in subcutaneous fat, epididymal fat, and peri-renal fat

The amounts of subcutaneous fat, epididymal fat, and peri-renal fat were measured after 8 weeks of treatment and necropsy and are shown in Table 6 below.

TABLE 6

| | Group 1 | Group 2 | Group 3 |
|---|---|---|---|
| | (ND) | (HFD) | (HFD + F) |
| Subcutaneous fat (g) | 0.33 | 1.53 | 1.05 |
| Epididymal fat (g) | 0.59 | 2.57 | 2.06 |
| Peri-renal fat (g) | 0.20 | 1.02 | 0.82 |

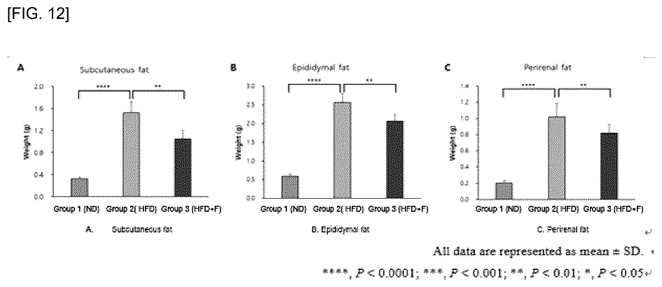

As shown in Table 6 and FIG. 12 above, the amount of body fat in Group 3 was significantly (P<0.01) reduced by 31.7% for subcutaneous fat, 19.9% for epididymal fat, and 19.6% for peri-renal fat compared to the amount of body fat in Group 2. Thus, it can be seen that probiotic complex F (LP02+LPL53) has a significantly pronounced effect on improving body fat reduction in mice.

### [Accession Number]

Name of depositary authority: Korea Research Institute of Bioscience and Biotechnology

Accession number: KCTC 14808BP (BCC-LP-02)
KCTC 14809BP (BCC-LPL-53)

Accession date: Dec. 6, 2021

### INDUSTRIAL AVAILABILITY

Composition comprising the Lactobacillus paracasei BCC-LP-02 strain, the Lactobacillus plantarum BCC-LPL-53 strain, or a mixture of the strains or cultures thereof of the present invention has an excellent body fat reduction effect by inhibiting adipocyte differentiation and intracellular fat accumulation through decreasing fatty acid (FA) concentration, producing exopolysaccharide (EPS), inhibiting lipase enzyme activity, and inhibiting glucose absorption.

In addition, it has been shown to prevent or treat obesity or obesity-related diseases by inhibiting fat absorption, inhibiting carbohydrate absorption, and inhibiting adipocyte differentiation in the small intestine or digestive tract.

Moreover, it has been shown to prevent or treat obesity or obesity-related diseases by reducing weight gain, reducing food efficiency, and reducing body fat.

1. A method for reducing body fat, comprising administering a composition comprising Lactobacillus paracasei strain BCC-LP-02 with accession number KCTC14808BP or a culture thereof.

2. A method for reducing body fat, comprising administering a Lactobacillus plantarum strain BCC-LPL-53 with accession number KCTC 14809BP or a culture thereof.

3. The method according to claim 1, in which the strain has a 16S rRNA sequence of SEQ ID NO: 1.

4. The method according to claim 2, in which the strain has a 16S rRNA sequence of SEQ ID NO: 2.

5. The method according to claim 1 or 2, in which the strain exhibits one or more characteristics selected from the group consisting of:
reduced fatty acid (FA) concentration;
exopolysaccharide (EPS) generation:
lipase enzyme inhibitory activity:
poor glucose absorption; and
inhibitory activity of preadipocyte differentiation.

6. A method for reducing body fat, comprising administering a composition comprising Lactobacillus paracasei strain BCC-LP-02 with accession number KCTC14808BP or a culture thereof and/or Lactobacillus plantarum strain BCC-LPL-53 with accession number KCTC 14809BP or a culture thereof.

7. The method according to claim 6, wherein the BCC-LP-02 strain and the BCC-LPL-53 strain exhibit a synergistic effect in a co-culture of the BCC-LP-02 strain and the BCC-LPL-53 strain, which maintains a complementary relationship in the utilization of metabolome (glyceric acid, malic acid, orotic acid, lactose) and helps growth.

8. The method according to claim 6, wherein the composition comprises the BCC-LP-02 and BCC-LPL-53 strains.

9. A method for the prevention or treatment of obesity, comprising administering a composition comprising Lactobacillus paracasei strain BCC-LP-02 with accession number KCTC14808BP or a culture thereof and/or Lactobacillus plantarum strain BCC-LPL-53 with accession number KCTC 14809BP or a culture thereof.

10. The method according to claim 9, wherein when administered to an individual, the composition reduces the rate of weight gain, reduces food efficiency, or reduces body fat.

11. The method according to claim 10, wherein the composition has the effect of inhibiting fat absorption, inhibiting carbohydrate absorption, and/or inhibiting adipocyte differentiation in small intestinal cells or the digestive tract.

12. A method for the prevention or amelioration of obesity, comprising administering a dietary supplement composition comprising Lactobacillus paracasei strain BCC-LP-02 with accession number KCTC14808BP or a culture thereof and/or Lactobacillus plantarum strain BCC-LPL-53 with accession number KCTC 14809BP or a culture thereof.

13. A method for the prevention or amelioration of obesity, comprising administering a quasi-drug composition comprising Lactobacillus paracasei strain BCC-LP-02 with accession number KCTC14808BP or a culture thereof and/or Lactobacillus plantarum strain BCC-LPL-53 with accession number KCTC 14809BP or a culture thereof.
